(19) United States
(12) Patent Application Publication
(10) Pub. No.: US 2013/0195815 A1
(43) Pub. Date: Aug. 1, 2013

(54) METHOD FOR LABELING INSULIN-SECRETING PANCREATIC BETA CELLS AND OBTAINING PURIFIED INSULIN-SECRETING PANCREATIC B-CELLS AND AN INSULIN REPORTED

(71) Applicant: NORTH CAROLINA CENTRAL UNIVERSITY, Durham, NC (US)

(72) Inventors: Jonathan Z. SEXTON, Durham, NC (US); Jay BRENMAN, Durham, NC (US)

(73) Assignee: NORTH CAROLINA CENTRAL UNIVERSITY, Durham, NC (US)

(21) Appl. No.: 13/651,005

(22) Filed: Oct. 12, 2012

Related U.S. Application Data
(60) Provisional application No. 61/546,577, filed on Oct. 13, 2011.

(51) Int. Cl. C12Q 1/68; A61K 35/39

(52) U.S. Cl. CPC C12Q 1/6897; A61K 35/39
USPC 424/93.21; 435/6.1; 435/6.13

(57) ABSTRACT

A method for labeling insulin-secreting pancreatic β-cells in human islets by using a genetically-encoded preproinsulin fluorescent protein reporter which targets an insulin fusion protein to correct insulin vesicles. The reporter system comprising an insulin and fluorescent protein construct provides real time tracking of secretory granules in live cells and allows for the accurate measuring of the level of secretion. The labeled cells are sorted by fluorescence-activated cell sorting to obtain purified insulin-secreting pancreatic β-cell pools from human islets. The β-cell pools are suitable for transplantation into the pancreas of diabetics in order to treat diabetes.

### CROSS REFERENCE APPLICATION

This application claims the benefit of U.S. Provisional Patent Application 61/546,577 filed Oct. 13, 2011, the content of which is incorporated herein by reference.

### FIELD OF THE INVENTION

This invention relates to a method for labeling insulin-secreting pancreatic β-cells and obtaining purified insulin-secreting pancreatic β-cells from mammalian pancreatic islets of Langerhan. The islets of Langerhan are the regions of the pancreas that are responsible for insulin secretion. This invention also relates to a reporter system for live-cell tracking of insulin dynamics in live cells. This invention also relates to a method for treating diabetes.

### BACKGROUND OF THE INVENTION

Diabetes is a major health problem which affects approximately 8% of the U.S. population resulting in roughly $178 billion annually in U.S. health-care associated costs (1). The current prediction is that one third of the U.S. population will develop T2D in their lifetime (1, 2). β-cells of the pancreatic islets secrete insulin in response to glucose and other nutrients. Diabetes results from the selective loss of β-cells in the pancreas which are responsible for producing insulin. Type-1 (T1D) diabetes occurs due to an autoimmune response from the individual which attacks insulin-secreting β pancreatic cells (11). Type-2 (T2D) diabetes occurs due to a loss of insulin-secreting Beta pancreatic cells due to factors such as obesity, sedentary lifestyle, insulin resistance, hyperinsulinemia, hyperglycemia, and elevated fatty acids which lead to cellular stress or toxic cellular environments that result in a decrease in β-cell number and function (12). Insulin deficiency in both T1D and T2D is characterized by a significant reduction in β-cell mass. The combination of hyperglycemia and hyperlipidemia is referred to as glucolipotoxicity, and is most common in patients with T2D. The exact mechanism(s) of glucolipotoxicity in human islets is unclear (7, 8, 9), but involves induction of endoplasmic reticulum (ER) stress, increased apoptosis and decreased islet function (9, 10). Apoptosis of the β-cells is an underlying cause of all diabetes, resulting in a complete loss or considerable reduction in insulin production (11, 12). Diabetes also causes debilitating and costly complications including neuropathy, nephropathy, and increases risks for limb amputation and other diseases including cardiovascular disease. Therapies that could protect against β-cell apoptosis and/or induce β-cell regeneration will open new avenues for the treatment of diabetes.

The pancreatic islets of Langerhan are multi-cellular aggregates composed of the following endocrine cells; glucagon producing Alpha cells, insulin producing B-cells, somatostatin producing Delta cells, pancreatic polypeptide producing (PP) cells, and ghrelin producing Epsilon cells (3, 5). These cell types exist within human islets in an approximate composition of 70% B-cells, 20% Alpha cells, 40% Gamma cells, <5% PP cells and 4% Epsilon cells (3, 4, 6). However, islet cell composition varies depending upon the location within the pancreas.

The main pharmacological approach for treating T1D is insulin injection. For T2D, there is primarily a focus on the use of insulin sensitizers (e.g. metformin and thiazolidinediones (TZDs)), incretin mimetics (e.g. GLP1R agonists such as Exenatide), DPPIV inhibitors (to raise endogenous levels of active GLP1) and various insulin secretagogues, as well as insulin injection. Despite these different classes of drugs for the treatment of T2D, only ˜36% of diabetic patients achieve the A1c goal of ≦7% (13). In addition, TZDs are associated with weight gain, an undesirable side effect; and metformin is not effective in all patients, and can lead to lactic acidosis. Several lines of evidence suggest activation of GLP1R signaling pathway can help preserve islet mass through stimulation of β-cell proliferation and protection from glucolipotoxicity (14, 15). However, the current GLP1 agonists are peptides that require injection, and recent attempts to develop small molecule agonists of the GLP1R have been unsuccessful in reaching the clinic. Furthermore the effects of GLP1R activation in humans appear limited (15), and it has been difficult to assess changes in β-cell mass in humans. Thus, current therapies do not effectively target the underlying loss of β-cell mass. There is significant need to develop therapies that can: 1) protect β-cells from glucolipotoxicity induced apoptosis, and 2) increase β-cell mass via stimulation of β-cell/islet growth.

Human islets are the most physiologically relevant system for the validation of potential therapies that modulate the metabolic regulation of insulin production as well as factors that regulate neogenesis, differentiation, and apoptosis of β-cells. However, the ability to identify and assess β-cell mass in islets in vivo and in vitro has proven difficult and there has been no efficient and efficacious method developed to obtain purified insulin producing β-cells from islets.

The isolation of large quantities of β-cells is innovative and has several advantages over current cell-based models for β-cell drug discovery. β-cells derived from primary human islets are clearly advantageous over existing rodent cell models and emerging immortalized β-cell lines (22, 23). Discovery of compounds in a phenotypic β-cell proliferation assay using primary non-cancerous\non-immortalized β-cells is advantageous because positive compounds from this assay will have substantially higher probability of translating to therapeutic effects in humans.

It is desired to create a method for labeling insulin producing β-cells in human islets which sufficiently targets insulin producing β-cells. This provides improved localization and signal intensity.

Furthermore, it is desired to have a reporter system which enables live-cell tracking of secretory granules in live cells. Current approaches rely on systems using rodent or porcine-derived islets (16). However, these islets do not accurately reflect physiological responses observed in human islets (17). A more relevant model for drug discovery efforts would utilize human derived β-cells, and ideally those that could be expanded and\or cryopreserved to ensure consistency and reduce variability in an assay platform.

Therefore, it is desired to provide a reporter that exhibits behavior identical or similar to an endogenous compound which can track secretory granules such as insulin vesicles within the cells. This can be used to accurately measure the level of secretion.

Accordingly, an aspect of the present invention is a method for labeling live insulin-secreting pancreatic β-cells in human islets.

A further aspect of the invention is a reporter system which tracks secretory granules in live cells and measures the level of secretion.

Another aspect of the present invention is to provide a method for obtaining purified insulin-secreting pancreatic β-cells from human islets.

Another aspect of the invention is β-cell specific fluorescent protein reporter driven by the insulin promoter to yield a fluorescent signal that is proportional to insulin synthesis, co-localizes with endogenous insulin and undergoes glucose-stimulated insulin secretion (GSIS).

Still a further aspect of the present invention is a method for treating diabetes.

### SUMMARY OF THE INVENTION

An aspect of the present invention is a method for identifying insulin-secreting pancreatic β-cells in human islets by using a genetically-encoded fluorescent protein reporter. Preproinsulin-mCherry fluorescent protein reporter (PPI-mCh) is such a genetically encoded fluorescent protein reporer. The PPI-mCh gene is introduced into insulin secreting cells at the genetic level, which causes the cells to produce fluorescent protein. This links insulin secretion to the production of the fluorescent protein wherein the measurement of fluorescence indicates the level of insulin synthesis in the cell.

The present invention is also directed to a reporter system comprising an insulin and preproinsulin fluorescent protein construct which provides real time tracking of secretory granules in live cells of mammals, preferably humans and allows for the quantitative measurement of the level of secretion. The present invention is also directed to a method for obtaining purified insulin-secreting pancreatic β-cells from human islets by using a preproinsulin reporter fluorescent protein reporter on human islets to label the insulin-producing β-cells and obtaining purified insulin-producing β-cells pools by using fluorescence-activated cell sorting. In addition as aspect of the invention is a method for treating diabetes by transplanting insulin-producing β-cells into the pancreas of a diabetic or patients with pre-diabetes that have impaired insulin secretion due to selective loss of β-cells.

### DESCRIPTION OF THE DRAWINGS

FIG. 1 is a graphical map of the vector insert containing the PreProInsulin gene flanked by the mCherry fluorescent reporter fusion protein.

FIG. 2 is a photograph field of pancreatic β-cells (INS-1 cell line) stably transfected with the insulin mCherry reporter.

FIG. 3(A) shows a comparison of a cluster of INS-1 cells stably tranfected with the mCherry insulin-reporter system.

FIG. 3(B) shows immunostaining of endogenous insulin granules with Alexa-488 conjugated anti-mouse insulin antibody.

FIG. 3(C) shows extensive colocalization of reporter with endogenous insulin showing mixed endogenous and labeled insulin granules.

FIG. 3(D) shows a field of stably transfected INS-1 cells showing distinct populations of insulin content within the cell in membrane associated and cytoplasmic pools.

FIG. 4 is a flow cytometry analysis of PPI-mCherry in INS-1 cells showing a 24.8 percent PPI-mCherry positive population.

FIG. 5(A) is a flow cytometry analysis of a mixture of cells carrying the reporter and cells without the reporter demonstrating feasibility for sorting cells based on fluorescent signal.

FIG. 5(B) showing a pure population of virally transduced INS-1 cells after sorting demonstrating a yield of over 90% pure β-cells.

FIG. 6 is a graph showing validation of the mCherry-insulin reporter demonstrating glucose and exenatide (Ex) potentiated insulin secretion. The Graph measuring relative fluorescent units of secreted mCherry-insulin is in dot-blot inset. (HG—high glucose (25 mM): LG—low glucose (5.6 mM): Ex—10 nM: NF—nifedipine 10 μM, calcium channel blocker/inhibitor) (Y-axis=normalized fluorescent units—FLU).

FIG. 7 is a schematic illustrating a process for producing pure, viable human β-cells.

FIG. 8 is a confocal imaging of an entire mouse pancreatic thin-section.

FIG. 9 is a co-localization of PPI-mCherry with insulin in INS-1 cells and human islets. The yellow to orange color indicates a high co-localization of PPI-mCherry (red) and insulin vesicles (green) in the INS-1 cells (A). In the human islets, glucagon (green) was not co-localized (B) whereas insulin (green) co-localized with PPI-mCherry reporter (C). (nuclei=blue).

FIG. 10(A) is a flow cytometry histogram for primary human sorting.

FIG. 10(B) is an image of an entire well of 3-day post-sorted primary human β-cells in 384-well format. The insert shows magnified field of blue nuclei and red PPI-mCherry signal with high purity and viability.

### DETAILED DESCRIPTION OF THE INVENTION

Before describing the present invention in detail, it has to be understood that this invention is not limited to particular embodiments described in this application. It is also to be understood that the terminology used herein is for the purpose of describing particular embodiments only, and is not intended to be limiting.

As used herein, the terms “treatment” and “therapy” and the like refer to alleviating, slowing the progression, prophylaxis, attenuation or cure of existing disease (e.g., diabetes).

It should also be noted that, as used in this specification and the appended claims, the singular forms “a,” “an,” and “the” include the plural unless the content clearly dictates otherwise.

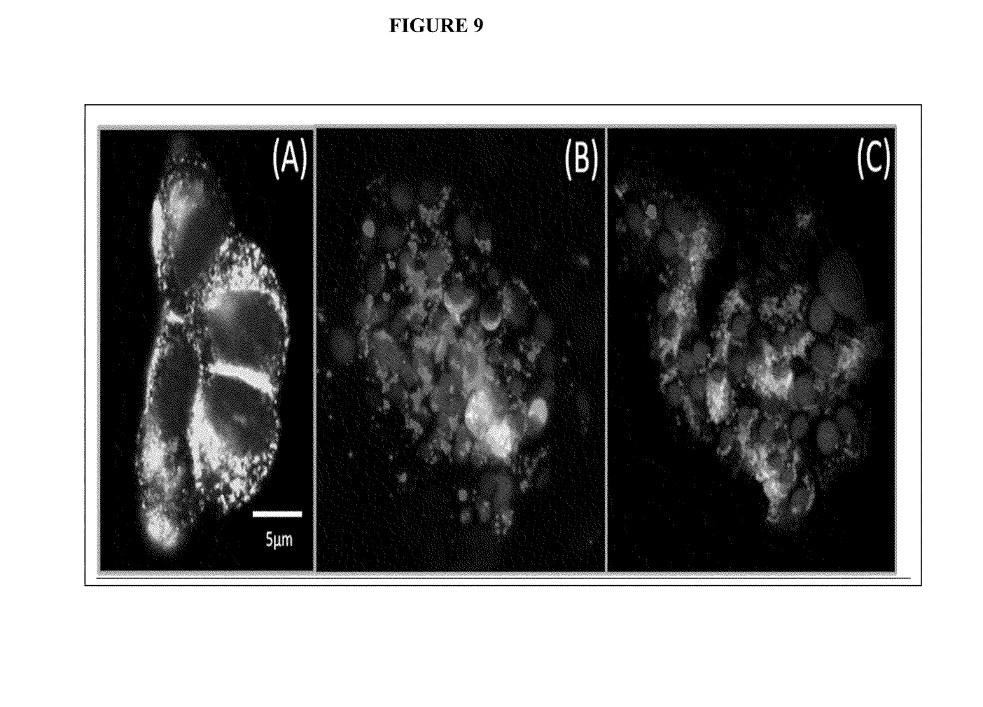

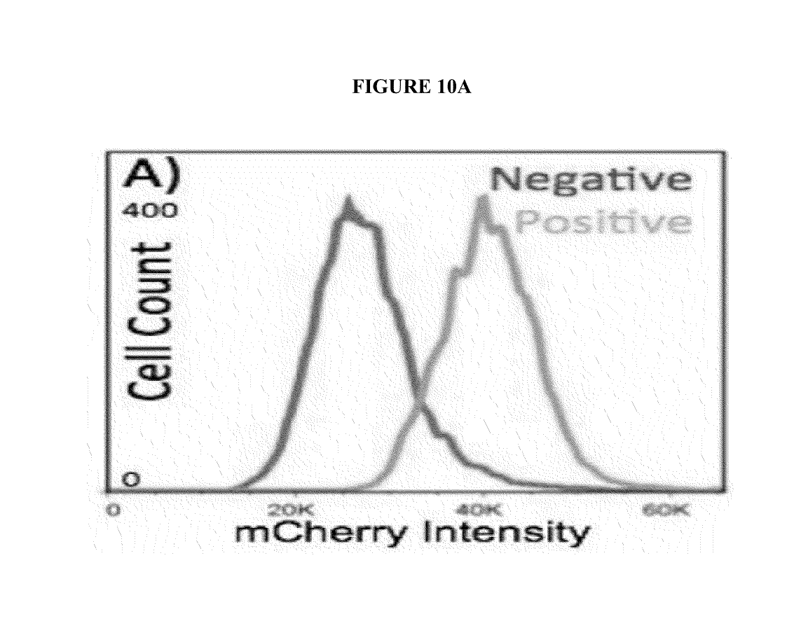

The method is directed to use of a genetically-encoded fluorescent preproinsulin reporter that labels endogenous insulin granules in live cells with a fluorescent protein. Insulin granules are bundles of insulin that are packaged and maintained in a condition for immediate release into the blood stream. The preproinsulin m-Cherry (PPI-mCh) reporter system is adapted to be under the control of the native human insulin promoter which is over 1000-times higher in β-cells than in other islet cell types. When this reporter system is under the control of an insulin promoter, the reporter exhibits a high level of colocalization (Pearson Coefficient above 0.95) and only the insulin-secreting β-cells in a pancreatic islet will become fluorescent. Inclusion of the signal peptide and other sequences ensures accurate targeting of the insulin fusion protein to the correct insulin secretory vesicles. A previous study generated an insertion of GFP within the C peptide of preproinsulin that reproduced localization and release of endogenous insulin (18). The reporter was optimized by replacing GFP with mCherry (less pH sensitive), as insulin vesicles undergo acidification during maturation, and through optimization of the arrangement and inclusion of C-peptide portion. The constructs are packaged in a pLenti delivery system and the reporter signal is driven by the mouse insulin promoter. Expression of this construct fluorescent insulin reporter preproinsulin-mCherry (PPI-mCherry) is β-cell specific and, due to the high conservation between species, works in both rodent and human cells. The 3′-UTR region of the preproinsulin mRNA is included, as this has been shown to confer a glucose-induced increase in mRNA stability. This reporter has been validated for efficient viral transduction in rat INS-1 cells and primary human pancreatic islets. Images in FIG. 9 show excellent co-localization between immunostained endogenous insulin and PPI-mCherry in rat INS-1 cells, as well as discrete labeling of β-cells in primary human islets that are co-stained for glucagon or endogenous insulin.

The mCherry fluorescent protein marker is stable at low pH (between pH 4-7) which is important since insulin granules undergo acidification to about 4.5 pH as they mature. While the use of the mCherry fluorescent protein marker is specifically described it is understood that other markers having similar pH stability and positive attributes for labeling preproinsulin may be used. Other fluorescent markers can be used.

In an aspect of the invention, the preproinsulin reporter is packaged in a pLenti delivery system (ptk-208 backbone) and operates under the control of the human insulin promoter which results in strong mCherry fluorescence intensity only in β-cells, where insulin promoter activity is present. While a pLenti delivery system (ptk-208 backbone) system is described, other commercially available lentivirus gene delivery systems may be used. The pLenti system for viral transduction of the reporter gene is adapted so that it will be easily translatable to primary human cell culture. The system preferably is also adapted to have an optimal multiplicity of infection. In one embodiment, Transdux reagent (System Biosciences Inc. Mountain View, Calif.) is incubated with cells and viral particles to achieve a higher efficiency of transduction. In addition, pLenti destination vectors (Invitrogen, Carlsbad, Calif.) may be used which are specifically optimized for high transduction efficiency with human cell lines. While the use of the pLenti system is specifically described, in alternative embodiments, other gene delivery systems known in the art may be used such as lipid-based transfection techniques. An insulin promoter is also required. It is preferred that the promoter sequence used is optimized to yield the greatest fluorescence differential between β-cells and other islet cell types in the α/β co-culture. The fluorescence differential is calculated by measuring the difference in fluorescent intensity between beta and non O-cells.

In an embodiment the PPI-mCh reporter is encoded to target insulin granules with high-specificity which ensures accurate targeting of the insulin fusion protein to the correct insulin vesicles with no loss of fluorescent signal during vesicle maturation. In a preferred embodiment the targeted sequence is as follows:

Sequence:
(SEQ ID NO: 1)
atggccctgtggatgcgcttcctgcccctgctggccctgctcttcctctgggagtcccaccccacccaggcttttgtcaagcagca
cctttgtggttcccacctggtggaggctctctacctggtgtgtggggagcgtggcttcttctacacacccatgtcccgccgtgaagt
ggaggacccacaagtggcacaactggagctgggtggaggcccggatccaccggtcgccaccatggtgagcaagggcgagg
aggataacatggccatcatcaaggagttcatgcgcttcaaggtgcacatggagggctccgtgaacggccacgagttcgagatcg
agggcgagggcgagggccgcccctacgagggcacccagaccgccaagctgaaggtgaccaagggtggccccctgcccttc
gcctgggacatcctgtcccctcagttcatgtacggctccaaggcctacgtgaagcaccccgccgacatccccgactacttgaag
ctgtccttccccgagggcttcaagtgggagcgcgtgatgaacttcgaggacggcggcgtggtgaccgtgacccaggactcctc
cctgcaggacggcgagttcatctacaaggtgaagctgcgcggcaccaacttcccctccgacggccccgtaatgcagaagaag
accatgggctgggaggcctcctccgagcggatgtaccccgaggacggcgccctgaagggcgagatcaagcagaggctgaa
gctgaaggacggcggccactacgacgctgaggtcaagaccacctacaaggccaagaagcccgtgcagctgcccggcgccta
caacgtcaacatcaagttggacatcacctcccacaacgaggactacaccatcgtggaacagtacgaacgcgccgagggccgc
cactccaccggcggcatggacgagctgtacaagtag

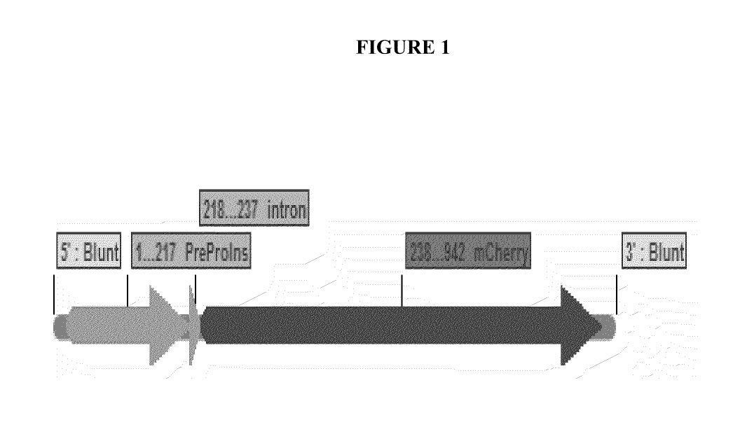

FIG. 1 shows a graphical map of the vector insert containing the PreProInsulin gene flanked by the mCherry fluorescent reporter fusion protein.

The delivery system may be implemented by transient transfections using mammalian protein expression vectors and lip-based transfection techniques. In an aspect of the invention, the PPI-mCh construct is cloned into a tet-inducible pLenti vector for transduction into primary human cells. An inducible system regulates gene expression in response to an external stimulus. The inducible system of the present invention allows the fluorescent signal to be turned on for the purposes of cell sorting and then be turned off for future use to maintain physiological integrity. The gene delivery system may also include helper plasmids co-transfected into the packaging cell line.

Another aspect is a method for tracking and monitoring the activity of secretory granules also referred to as “insulin vesicles” inside live pancreatic β-cells. A construct comprising insulin and a fluorescent protein by transfection as described above is used in this method.

The insulin and preproinsulin-mCherry fluorescent protein fusion construct is able to accurately track insulin vesicles within the cells in real time. The construct may be viewed by imaging means known in the art. The insulin and preproinsulin-mCherry fluorescent protein fusion construct behaves identically or substantially similar to endogenous insulin with respect to glucose stimulated insulin secretion and secretes fluorescently labeled insulin in an identical manner as endogenous insulin, with respect to glucose stimulated insulin secretion. Accordingly, the secretion fractions of the construct may be assayed for quantitative analysis. For instance, the secretions may be monitored in a cell culture media supernatant in order to accurately measure insulin secretion. Furthermore, the construct responds normally to drugs that affect insulin secretion.

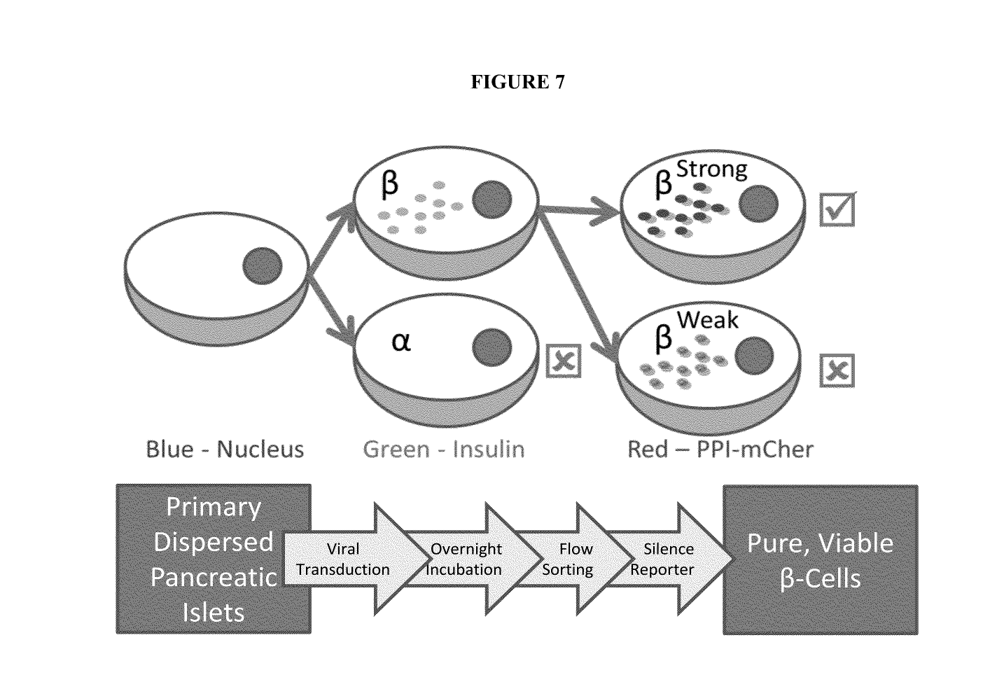

Another aspect of this invention is a method of obtaining purified insulin-secreting pancreatic β-cells from a human islet. As shown in FIG. 7, the method is comprised of the steps of labeling insulin-secreting β-cells in a human islet by use of a genetically encoded fluorescent preproinsulin reporter such as PPI-mCh delivered by a suitable delivery system as described and incorporated herein by reference. After viral transduction, the fluorescence activity of the reporter may be induced. The reporter inducible compound enables activation of the fluorescence signal. Other fluorescent protein reporters having suitable pH stability may be used including fruit colored fluorescent proteins. In an embodiment the reporter inducible compound is tetracycline.

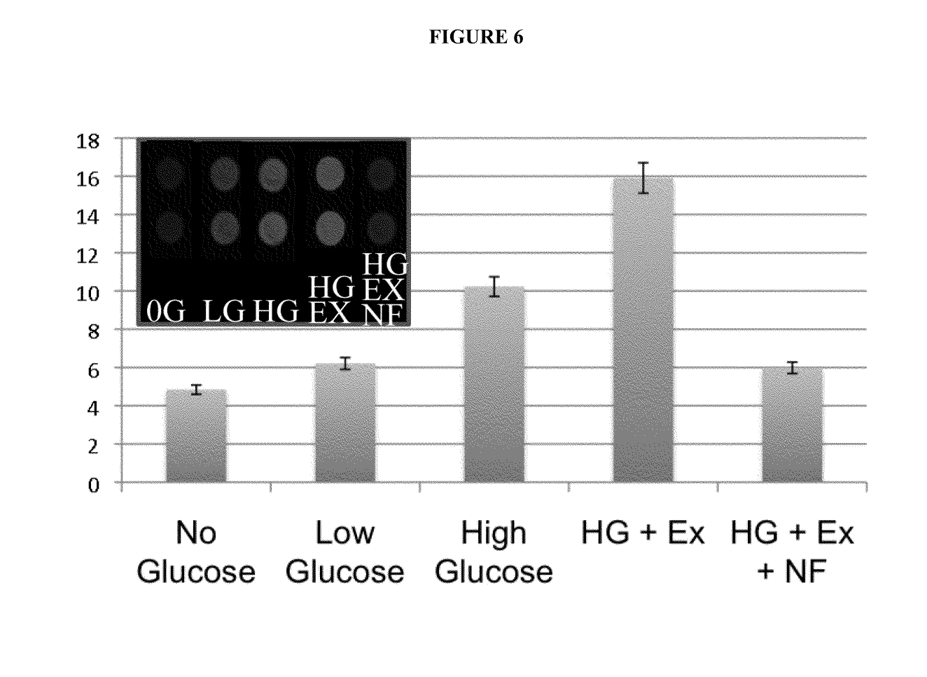

The reporter system was tested in insulin secretion assays. The reporter acts with a normal physiological response indicative of normal insulin regulation in this reporter. More specifically, FIG. 6 shows a graph measuring relative fluorescent units of secreted mCherry-insulin with dot-blot inset in conditions of no glucose, low glucose (5.6 mM), high glucose (25 mM), Exendin-4 (10 nM), and in the presence of nifedipine (10 μM), Exendin-4 (10 nM), and a calcium channel blocker/inhibitor. The graph indicates that glucose starvation causes a build-up of fluorescently-labeled cellular insulin. Nifedapine blocks the secretion of fluorescently-labeled insulin. Microscopic analysis indicates that the fluorescently-labeled insulin is mixed with endogenous insulin granules and is regulated by the same mechanisms. The new reporter cell lines secrete insulin in the same manner as the parental cell lines. The new cell lines secrete endogenous insulin in a typical manner and respond normally to drugs that affect insulin secretion.

After being labeled, the cells are sorted by fluorescence-activated cell sorting to obtain purified β-cells that have demonstrated competence for insulin secretion. The reporter inducible compound may then be removed to permanently silence the reporter so that the live β-cells function in the manner of a normal β-cell. The pool of viable and competent β-cells pools is able to be maintained under normal cell culture conditions and can be saved for future use. These cells are useful for research and development of treatments particularly with respect to diabetes. Potential uses for purified β-cells include: (1) use in genomic and proteomic experiments to identify differential gene and gene product regulation that can be used to identify cell-surface markers for immunologic differentiation of β-cells from other cell types; and (2) use for drug discovery purposes, to identify compounds/mechanisms that act to preserve/delay programmatic cell death (apoptosis) in β-cells, thereby preserving intact β-cell mass and halting diabetes progression to insulin dependence.

A further aspect of the present invention is a method for treating diabetes by transplanting into the pancreas of a patient with diabetes or a person at risk of developing diabetes an effective amount of insulin-producing β-cells.

It is understood that modifications that do not substantially affect the activity of the various embodiments of this invention are included within the invention disclosed herein. Accordingly, the following examples are intended to illustrate but not to limit the present invention.

### EXAMPLES

### Example 1

Colocalization of PPI-mCh Reporter on Rodent INS-1 Cell Lines

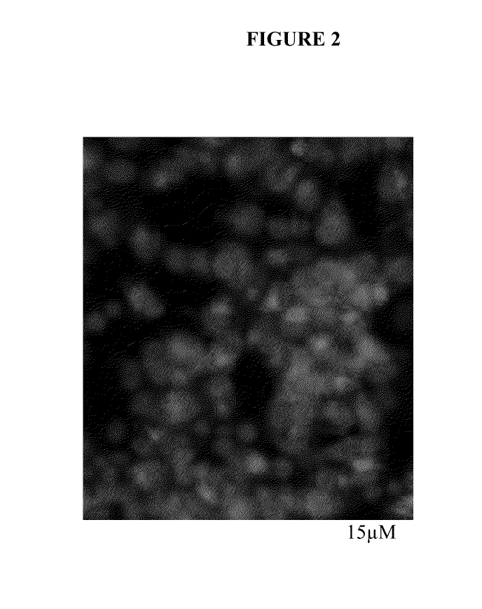

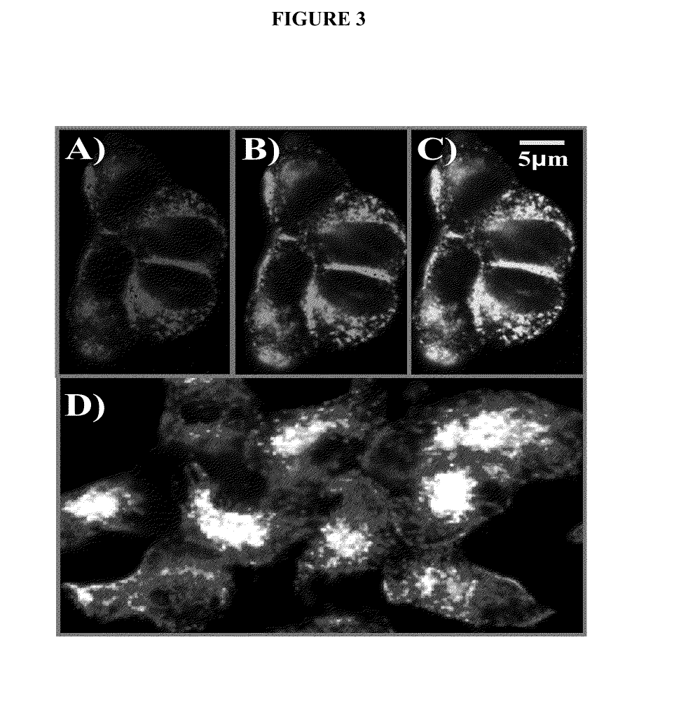

The PPI-mCh reporter was transfected on rodent insulimona INS-1 cell lines capable of glucose-stimulated insulin secretion. The PPI-mCh reporter exhibits a high degree (Pearson's coefficient ≧0.95) of colocalization between the fluorescent protein reporter and endogenous insulin. As shown in FIG. 3(c), immuno-colocalization is displayed at 600× magnification between mCherry red labeled insulin vesicles (shown in red) and Alexa-488 conjugated anti-insulin antibody in the INS-1 cell line as indicated by the significant presence of the color yellow. FIG. 2 shows a field of insulin-mCherry stably-transfected cells. The red color indicates insulin vesicles, and the blue color indicates nuclei.

### Example 2

### Reporter System Construct in GSIS Assays

GSIS: INS-1 cells were transduced with PPI-mCherry and allowed to recover. The cells were stepped-down in low glucose buffer for 1 h and then incubated for 2 h in buffer containing no glucose, low glucose, high glucose or high glucose+exenatide. Conditioned media was removed and spotted onto a membrane and relative fluorescence determined using an Odyssey CLx imager (Licor Corp). The parental INS-1 cells used in the initial studies have historically shown a 2-fold GSIS (19). Consistent with this a 1.7-fold (high vs low glucose) glucose stimulated release of the PPI-mCherry reporter (FIG. 2) was observed. Exenatide (10 nM) displayed a synergistic response with high glucose (2.7-fold compared to low glucose). This response was inhibited by the calcium channel blocker nilfedipine. These data suggest that PPI-mCherry undergoes GSIS and does not interfere with secretion of endogenous insulin (not shown).

### Example 3

### Preliminary High Content Assay Development

Multivariate, high-content assay development for β-cell proliferation involves seeding purified β-cells per well in 384-well plates, incubating with 5 mL compounds for 72 h and the plates are stained with 10 μg/mL Hoechst-33342 and 50 nM YoYo-1 iodide for 45 minutes. Automated microscopy is used to capture 3-color multiplexed images for each well; nuclei (Blue—Hoechst-33342-cell permeant nucleic acid stain), viability (Green—YoYo-1 iodide, cell impermeant nucleic acid stain), and insulin (Red—PPI-mCherry). Image analysis is subsequently performed to tabulate the number of cells/well, live:dead score based on percentage of YoYo-1 positive cells, and β-cell confirmation by mCherry. A proliferation score is calculated by comparing the number of cells in compound treated wells vs. the negative control wells. The cellular insulin content in the feature extraction process that will be analyzed during hit triage and selection can be tabulated. One of the many benefits of high-content screening is the ability to look at the images of treated cells selected by numerical scoring systems and to verify effects and rule out false-positives.

### Example 4

### β-Cell Sorting

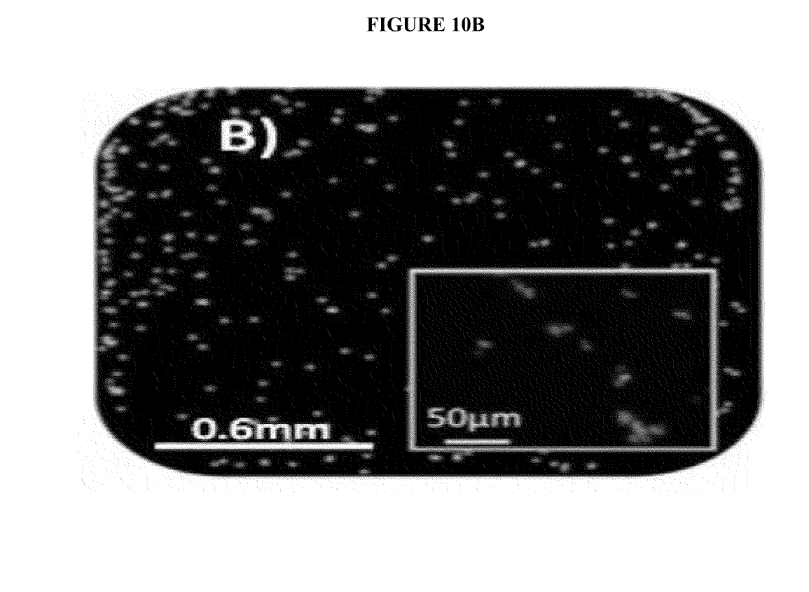

Purified human islets were dispersed into a single cell suspension by brief digestion with trypsin, induced with lentivirus constructs and allowed to recover overnight. Maximum fluorescent signal intensity was observed at 72 h, and flow sorting was performed to isolate mCherry positive cells. FIG. 10A shows adequate separation for sorting in the mCherry channel. FIG. 10B shows whole well imaging for high content screening of the sorted β-cells, consisting of a 5×5 image montage for high resolution cell counting and feature extraction. Post sorted β-cells were found to be highly viable after 3 days in culture (>95%), and were over 85% pure. The maximum theoretical yield for β-cells from dispersed islets is ˜70%. Approximately 50% yield was obtained from a small sort batch of 250 k dispersed cells yielding ˜140 k β-cells.

### Example 5

### Efficiency of Viral Transduction by Delivery System

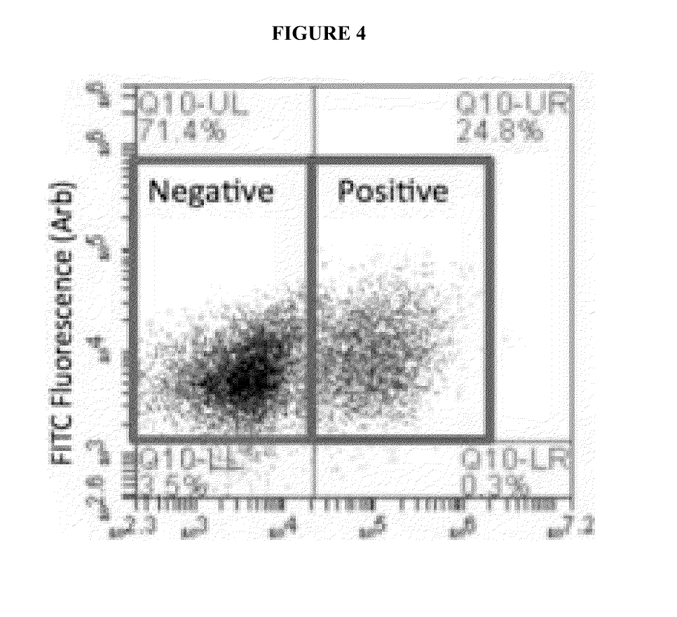

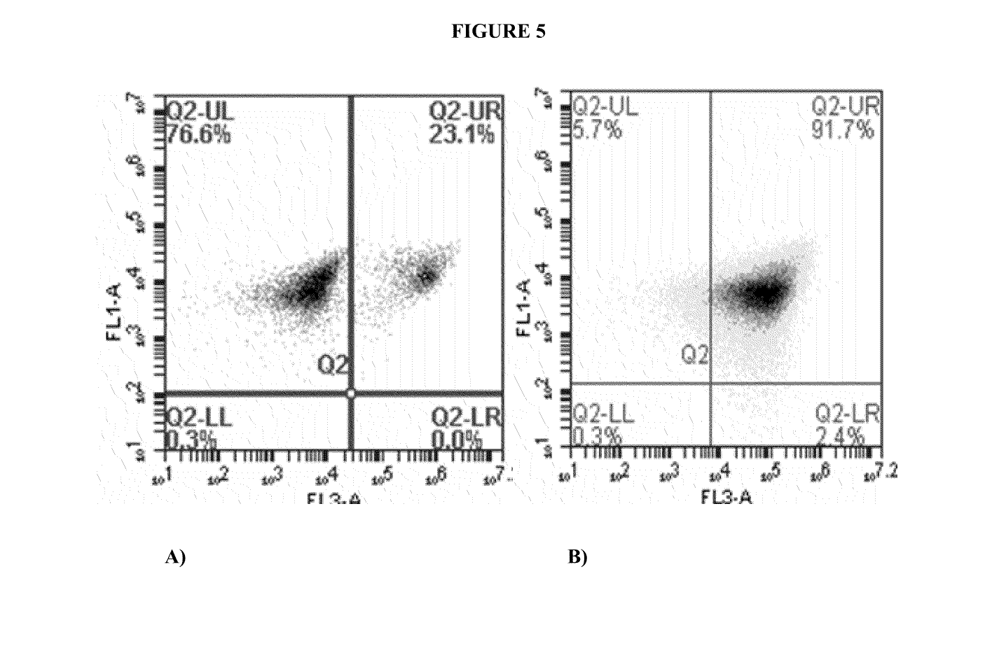

As shown in FIGS. 4 and 5, a flow cytometry analysis of initial viral transduction with a Multiplicity of Infection (MOI) equal to 1 in a rodent INS-1 cell line indicates a 24.8 percent PPI-mCh positive population.

As low cytometry experiments demonstrate that the method serves to effectively sort β-cells from non-β-cells by the presence of the reporter which is indicative of insulin secretion capacity. FIG. 5 shows a mixed population of cells carrying the reporter and cells that do not carry the reporter. There is a clear separation of the population on the X-axis for the cells. The cells that contain the reporter cluster on the right while cells that do not contain the reporter cluster on the left.

### Example 6

### Labeling of Pancreatic Cells

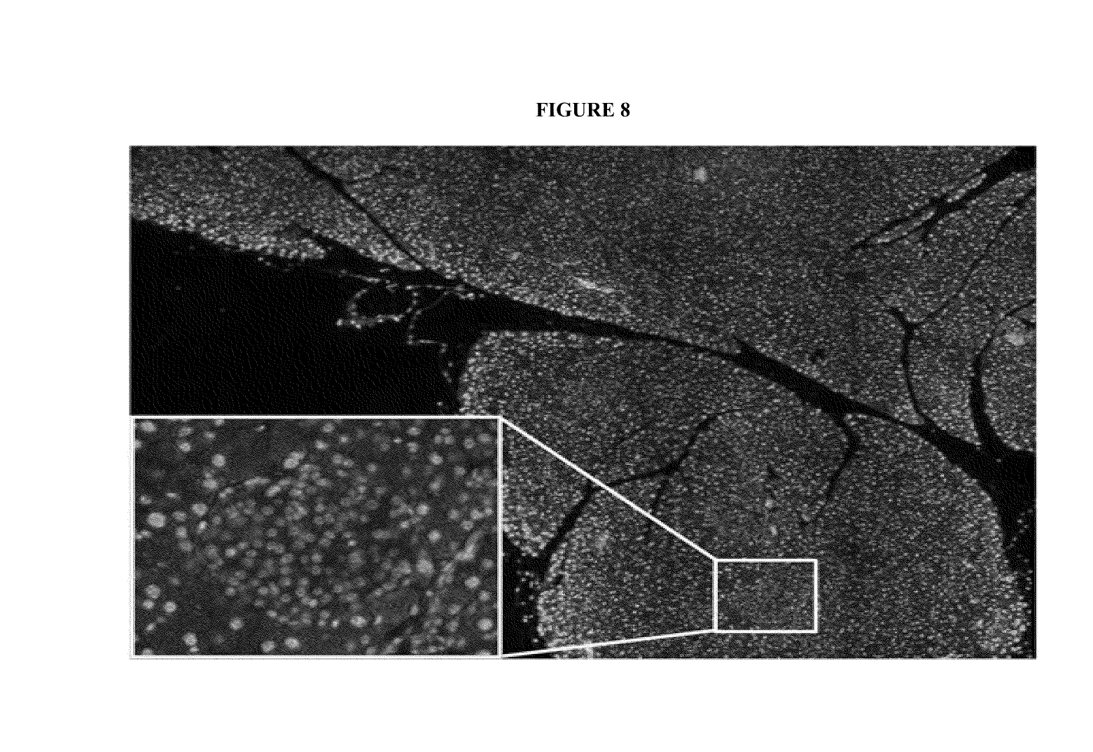

FIG. 8 shows a thin section of an entire mouse pancreas with differential labeling of α and β-cells using high-resolution confocal imaging. The imaging reveals an accurate sub-cellular localization of insulin granules.

### REFERENCES

1. Centers for Disease Control (2007) National diabetes fact sheet: general information and national estimates on diabetes in the United States (Atlanta, Ga.), (Prevention CfDCa).
2. NIDDK NDlC (2007) National Diabetes Statistics 2007.
3. Collombat P, Xu X, Heimberg H, & Mansouri A (2010) Pancreatic beta-cells: from generation to regeneration (Translated from eng) Semin Cell Dev Biol 21(8):838-844 (in eng).
4. Cabrera 0, et al. (2006) The unique cytoarchitecture of human pancreatic islets has implications for islet cell function. (Translated from eng) Proc Nall Acad Sci USA 103(7):2334-2339 (in eng).
5. Andralojc KM, et al. (2009) Ghrelin-producing epsilon cells in the developing and adult human pancreas. (Translated from eng) Diabetologia 52(3):486-493 (in eng).
6. Elayat A A, el-Naggar M M, & Tahir M (1995) An immunocytochemical and morphometric study of the rat pancreatic islets. (Translated from eng) J Anat 186 (Pt 3):629-637 (in eng).
7. Poitout V. Glucolipotoxicity of the pancreatic beta-cell: myth or reality? Biochem Soc Trans. 2008; 36(Pt 5):901-4. PMCID: 2889238.
8. Sargsyan E, Bergsten P. Lipotoxicity is glucose-dependent in INS-1E cells but not in human islets and MIN6 cells. Lipids Health Dis. 2011; 10(1):115. PMCID: 3141549.
9. Cunha D A, Hekerman P, Ladriere L, Bazarra-Castro A, Ortis F, Wakeham M C, et al. Initiation and execution of lipotoxic ER stress in pancreatic beta-cells. J Cell Sci. 2008; 121(Pt 14):2308-18.
10. Poitout V, Robertson R P. Glucolipotoxicity: fuel excess and beta-cell dysfunction. Endocr Rev. 2008; 29(3):351-66. PMCID: 2528858.
11. Kloppel G, Lohr M, Habich K, Oberholzer M, Heitz P U. Islet pathology and the pathogenesis of type 1 and type 2 diabetes mellitus revisited. Surv Synth Pathol Res. 1985; 4(2):110-25.
12. Lupi R, Del Prato S. Beta-cell apoptosis in type 2 diabetes: quantitative and functional consequences. Diabetes Metab. 2008; 34 Suppl 2:S56-64.
13. Meece J. Dispelling myths and removing barriers about insulin in type 2 diabetes. Diabetes Educ. 2006; 32(1 Suppl):9S-18S.
14. Gunasekaran U, Gannon M. Type 2 diabetes and the aging pancreatic β-cell. Aging (Albany N.Y.). 2011; 3(6):565-75. PMCID: 3164365.
15. Cavener D R, Gupta S, McGrath B C. PERK in β-cell biology and insulin biogenesis. Trends Endocrinol Metab. 2010; 21(12):714-21. PMCID: 2991375.
16. Cernea S, Pozzilli P. New potential treatments for protection of pancreatic B-cell function in Type 1 diabetes. Diabet Med. 2008; 25(11):1259-67. PMCID: 2701557.
17. Kharouta M, Miller K, Kim A, Wojcik P, Kilimnik G, Dey A, et al. No mantle formation in rodent islets—the prototype of islet revisited. Diabetes Res Clin Pract. 2009; 85(3):252-7. PMCID: 2746740.
18. Watkins S, et al. (2002) Imagining secretory vesicles by fluorescent protein insertion in propeptide rather than mature secreted peptide. (Translated from eng) Traffic 3(7):461-471 (in eng).
19. Muralidharan B, Bakthavachalu B, Pathak A, Seshadri V. A minimal element in 5′UTR of insulin mRNA mediates its translational regulation by glucose. FEBS letters. 2007; 581(21):4103-8.
20. Carpenter A E, et al. (2006) CellProfiler: image analysis software for identifying and quantifying cell phenotypes. (Translated from eng) Genome Biol 7(10):R100 (in eng).
21. Flotte T, et al. (2001) Efficient ex vivo transduction of pancreatic islet cells with recombinant adeno-associated virus vectors. (Translated from eng) Diabetes 50(3):515-520 (in eng).
22. Walpita D, Hasaka T, Spoonamore J, Vetere A, Takane K K, Fomina-Yadlin D, et al. A human islet cell culture system for high-throughput screening. J Biomol Screen. 2012; 17(4):509-18.
23. Ravassard P, Hazhouz Y, Pechberty S, Bricout-Neveu E, Armanet M, Czernichow P, et al. A genetically engineered human pancreatic β-cell line exhibiting glucose-inducible insulin secretion. J Clin Invest. 2011; 121(9):3589-97. PMCID: 3163974.
24. Shaner N C, Steinbach P A, Tsien R Y. A guide to choosing fluorescent proteins. Nat Methods. 2005; 2(12):905-9.
25. Orci L, Ravazzola M, Amherdt M, Madsen O, Perrelet A, Vassalli J D, et al. Conversion of proinsulin to insulin occurs coordinately with acidification of maturing secretory vesicles. J Cell Biol. 1986; 103(6 Pt 1):2273-81. PMCID: 2114621.
26. Tillmar L, Welsh N. Glucose-induced binding of the polypyrimidine tract-binding protein (PTB) to the 3′-untranslated region of the insulin mRNA (ins-PRS) is inhibited by rapamycin. Mol Cell Biochem. 2004; 260(1-2):85-90.
27. Tillmar L, Carlsson C, Welsh N. Control of insulin mRNA stability in rat pancreatic islets. Regulatory role of a 3′-untranslated region pyrimidine-rich sequence. The Journal of biological chemistry. 2002; 277(2):1099-106.
28. Lee E K, Kim W, Tominaga K, Martindale J L, Yang X, Subaran S S, et al. RNA-binding protein HuD controls insulin translation. Mol Cell. 2012; 45(6):826-35. PMCID: 3319250.
29. Wicksteed B, Uchizono Y, Alarcon C, McCuaig J F, Shalev A, Rhodes C J. A cis-element in the 5′ untranslated region of the preproinsulin mRNA (ppIGE) is required for glucose regulation of proinsulin translation. Cell metabolism. 2007; 5(3):221-7.
30. Wicksteed B, Herbert T P, Alarcon C, Lingohr M K, Moss L G, Rhodes C J. Cooperativity between the preproinsulin mRNA untranslated regions is necessary for glucose-stimulated translation. The Journal of biological chemistry. 2001; 276(25):22553-8.
31. Sasamoto H, Futami M, Ando Y, Nakaji S. Cryopreservation of rat islets of Langerhans by vitrification. J Artif Organs. 2012.

What is claimed is:

1. A method for labeling pancreatic β-cells that are viable and demonstrate competency secreting insulin from human islets comprising the steps of transfecting endogenous insulin granules in live cells with a preproinsulin-mCherry reporter (PPI-mCH) and detecting production of insulin.

2. The method of claim 1, wherein said reporter is packaged in a pLenti delivery system (ptk-208 backbone) and operates under the control of the CytoMegalovirus promoter.

3. The method of claim 1, wherein said reporter is cloned into a tet-inducible pLenti vector for transduction into primary human cells.

4. The method of claim 1, wherein preproinsulin-mCherry reporter (PPI-mCH) is adapted to target the following fragment:
SEQ ID NO: 1
Atggccctgtggatgcgcttcctgcccctgctggccctgctcttcctctgggagtcccaccccacccaggcttttgtcaagcagc
acctttgtggttcccacctggtggaggctctctacctggtgtgtggggagcgtggcttcttctacacacccatgtcccgccgtgaag
tggaggacccacaagtggcacaactggagctgggtggaggcccggatccaccggtcgccaccatggtgagcaagggcgag
gaggataacatggccatcatcaaggagttcatgcgcttcaaggtgcacatggagggctccgtgaacggccacgagttcgagatc
gagggcgagggcgagggccgcccctacgagggcacccagaccgccaagctgaaggtgaccaagggtggccccctgccctt
cgcctgggacatcctgtcccctcagttcatgtacggctccaaggcctacgtgaagcaccccgccgacatccccgactacttgaa
gctgtccttccccgagggcttcaagtgggagcgcgtgatgaacttcgaggacggcggcgtggtgaccgtgacccaggactcct
ccctgcaggacggcgagttcatctacaaggtgaagctgcgcggcaccaacttcccctccgacggccccgtaatgcagaagaa
gaccatgggctgggaggcctcctccgagcggatgtaccccgaggacggcgccctgaagggcgagatcaagcagaggctga
agctgaaggacggcggccactacgacgctgaggtcaagaccacctacaaggccaagaagcccgtgcagctgcccggcgcct
acaacgtcaacatcaagttggacatcacctcccacaacgaggactacaccatcgtggaacagtacgaacgcgccgagggccg
ccactccaccggcggcatggacgagctgtacaagtag.

5. A method for obtaining purified pancreatic β-cells that are viable and demonstrate competency secreting insulin from human islets comprising the steps of labeling endogenous insulin granules in live cells with a preproinsulin reporter (PPI-mCH) and sorting the cells by use of fluorescence-activated cell sorting.

6. A method for treating diabetes comprising the steps of transplanting into the pancreas of a diabetes patient an effective amount of insulin-producing β-cells obtained by the method of claim 5.

7. The method according to claim 5, wherein the measurement of fluorescence indicates the level of insulin synthesis in the cell

8. A method for real time tracking of insulin vesicles in live cells and measuring the level of secretion of insulin in the cells comprising the steps of:
transfecting endogenous insulin granules in live cells with a fluorescent protein reporter to form a construct which behaves like endogenous insulin;
inducing said fluorescent protein reporter; and
monitoring the presence of fluorescence from said reporter.

9. The method according to claim 8, wherein the live cells are pancreatic β-cells.

10. The method of claim 9, comprising the further step of measuring the level of secretion from the cells.


SEQUENCE LISTING (1 sequence)

SEQ ID NO: 1 - DNA, 945 bp, ARTIFICIAL SEQUENCE
  Target insulin
atggccctgtggatgcgcttcctgcccctgctggccctgctcttcctctgggagtcccaccccacccaggcttttgtcaagcagcacctttgtggttcccacctggtggaggctctctacctggtgtgtggggagcgtggcttcttctacacacccatgtcccgccgtgaagtggaggacccacaagtggcacaactggagctgggtggaggcccggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag